(19) United States
(12) Patent Application Publication
(10) Pub. No.: US 2025/0041327 A1
(43) Pub. Date: Feb. 6, 2025

(54) COMPOSITIONS FOR PREVENTING OR FOR REDUCING RISK OF DEVELOPING SECONDARY INFECTIOUS DISEASES

(71) Applicant: MEIJI CO., LTD., Tokyo (JP)

(72) Inventors: Hiroki ISHIKAWA, Tokyo (JP); Akira TAMURA, Tokyo (JP); Hiroshi KANO, Tokyo (JP); Shoko ISHII, Tokyo (JP)

(73) Assignee: MEIJI CO., LTD., Tokyo (JP)

(21) Appl. No.: 18/720,268

(22) PCT Filed: Dec. 21, 2022
(86) PCT No.: PCT/JP2022/046989

§ 371 Date: Jun. 14, 2024

(30) Foreign Application Priority Data
Dec. 22, 2021 (JP) 2021-208198

(51) Int. Cl. A61K 31/715; A61K 35/747; A61P 31/04

(52) U.S. Cl. CPC A61K 31/715; A61K 35/747; A61P 31/04

(57) ABSTRACT

The objects are to provide an active ingredient that can be ingested for prevention of secondary infectious diseases after viral infections on a daily basis at ease without risk of emergence of resistant bacteria, and to clarify the effect of EPS of lactic acid bacteria on secondary infectious diseases after viral infections. There is provided a composition for preventing a secondary infectious disease, e.g., pneumonia caused by Staphylococcus aureus, after viral infection, or for reducing the risk of developing the same, which contains an exopolysaccharide of lactic acid bacteria.

### TECHNICAL FIELD

The present invention relates to a composition for preventing a secondary infectious disease, especially a secondary bacterial infectious disease, after a viral infection, or for reducing the risk of developing those ones.

### BACKGROUND ART

The influenza viruses infect a large number of people each year, especially during the winter months, and despite the existence of vaccines and antiviral drugs, they remain a serious public health problem worldwide. Influenza virus infection not only causes direct symptoms of viral infection, such as sudden high fever, headache, and pharyngitis, but also predisposes people to secondary bacterial infections, which can cause severe and fatal pneumonia. Bacteria known to cause secondary bacterial infections after influenza virus infection include Staphylococcus aureus, Moraxella catarrhalis, Pseudomonas aeruginosa, and so forth. Among them, Staphylococcus aureus has recently come to be considered as a serious pathogen of influenza-associated pneumonia (Non-patent documents 1 and 2).

The increased risk of secondary bacterial infection during influenza virus infection is thought to be due to dysfunction of the airway-alveolar barrier caused by inadequate binding between tight junctions by ZO-1, occludin, caudin-1 and E-cadherin (Non-patent documents 3 and 4). In addition, it has been reported that infection with influenza viruses increases expressions of transmembrane and extracellular matrix proteins, such as intracellular adhesion molecule 1 (ICAM-1), carcinoembryonic antigen-related cell adhesion molecule 1 (CEACAM-1), fibronectin, and platelet-activating factor receptor (PAF-r), which are used as bacterial adhesion molecules (Non-patent documents 5 to 8).

By the way, use of lactic acid bacteria and their products for the prevention of infectious diseases, etc., has been examined. For example, Patent document 1 describes a composition for use in prevention or treatment of respiratory diseases of subjects, which contains Bifidobacterium lactis BL04 and/or fermentation product of Bifidobacterium lactis BL04, and/or cell lysate of Bifidobacterium lactis BL04. Patent document 2 describes a drug for treatment of bacterial infectious diseases in the gastrointestinal tract, which contains at least one kind of sugar-containing inhibitory substance, and describes that this drug may also be used for treatment of influenza, co-infections of bacteria and influenza viruses, or secondary bacterial infectious diseases associated with influenza, and polysaccharides derived from lactic acid bacteria are preferred as the sugar-containing inhibitory substance.

Lactobacillus delbrueckii ssp. bulgaricus OLL1073R-1 (OLL1073R-1) is known as a lactic acid bacterium that produces exopolysaccharide (EPS). It has been reported that oral administration of EPS derived from OLL1073R-1 to mice significantly improved the survival rate and reduced the amount of virus in the lungs after influenza virus infection compared with water-administered mice (Non-patent document 9). There has also been reported a mechanism of preventing influenza virus infection by EPS administration, according to which sufficient viral clearance is provided by activation of NK cells by IFN-γ, which is thought to be produced by splenocytes derived from mice administered with EPS, as innate immunity, and production of anti-influenza virus IgA and IgG1 antibodies in bronchoalveolar lavage fluid (BALF), as acquired immunity (Non-patent document 9 mentioned above and Non-patent document 10).

### PRIOR ART REFERENCES

### Patent Documents

Patent document 1: Japanese Patent Unexamined Publication (Kohyo) No. 2014-517003
Patent document 2: Japanese Patent Unexamined Publication (Kohyo) No. 2005-504017

### Non-Patent Documents

Non-patent document 1: Shah, N. S., et al. (2016) Bacterial and viral co-infections complicating severe influenza: Incidence and impact among 507 U.S. patients, 2013-14, J Clin Virol, 80, 12-19
Non-patent document 2: Jia, L., et al. (2018) Severe Pneumonia Caused by Coinfection With Influenza Virus Followed by Methicillin-Resistant Staphylococcus aureus Induces Higher Mortality in Mice, Front Immunol, 9, 3189
Non-patent document 3: Short, K. R., et al. (2016) Influenza virus damages the alveolar barrier by disrupting epithelial cell tight junctions, Eur Respir J, 47, 954-966
Non-patent document 4: Linfield, D. T., et al. (2021) Airway tight junctions as targets of viral infections, Tissue Barriers, 9, 1883965
Non-patent document 5: Watson, L., et al. (2013) Role of intercellular adhesion molecule-1 and polymeric immunoglobulin receptor in the co-colonization of airways with Streptococcus pneumoniae and Haemophilus influenzae, Pediatr Infect Dis J, 32, 1155
Non-patent document 6: Jiang, H., et al. (2017) Sphingosine 1-phosphate receptor 1 (S 1 PR 1) agonist CYM5442 inhibits expression of intracellular adhesion molecule 1 (ICAM1) in endothelial cells infected with influenza A viruses, PLoS One 12, e0175188
Non-patent document 7: Vimalanathan, S., et al. (2017) Prevention of influenza virus induced bacterial superinfection by standardized Echinacea purpurea, via regulation of surface receptor expression in human bronchial epithelial cells, Virus Res, 233, 51-59
Non-patent document 8: Zhao, J., et al. (2021) Liu Shen Wan inhibits influenza virus-induced secondary Staphylococcus aureus infection in vivo and in vitro, J Ethnopharmacol 277, 114066
Non-patent document 9: Nagai, T. et al. (2011) Effects of oral administration of yogurt fermented with Lactobacillus delbrueckii ssp. Bulgaricus OLL1073R-1 and its exopolysaccharides against influenza virus infection in mice, Int Immunopharmacol, 11, 2246-2250
Non-patent document 10: Makino, S., et al. (2016), Enhanced natural killer cell activation by exopolysaccharides derived from yogurt fermented with Lactobacillus delbrueckii ssp. bulgaricus OLL1073R-1, J Dairy Sci, 99, 915-923

### SUMMARY OF THE INVENTION

### Object to be Achieved by the Invention

Currently, the only effective method recognized for the prevention of secondary bacterial infectious diseases following viral infections is administration of antibiotics. There is concern that antibiotic administration generally leads to the emergence of resistant bacteria. It would be desirable to have an active ingredient for the prevention of secondary bacterial infectious diseases that can be ingested on a daily basis at ease without the risk of emergence of resistant bacteria.

On the other hand, it had not been clear whether the EPS of OLL1073R-1 affects secondary bacterial infectious diseases after viral infections.

### Means for Achieving the Object

The present invention provides the followings.

[1] A composition for preventing, or for reducing risk of developing a secondary infectious disease, especially a secondary bacterial infectious disease, after a virus infection, which contains an exopolysaccharide of lactic acid bacteria.
[2] The composition according to 1, wherein the virus is any of viruses that cause common cold syndrome.
[3] The composition according to 1 or 2, wherein the bacterial infectious disease is one caused by any selected from the group consisting of Staphylococcus aureus, Moraxella catarrhalis, Pseudomonas aeruginosa, Haemophilia influenzae, Klebsiella pneumoniae, Streptococcus pneumoniae, and glucose-nonfermentative gram-negative rods.
[4] The composition according to any one of 1 to 3, wherein the lactic acid bacteria are those classified as Lactobacillus delbrueckii subspecies bulgaricus.
[5] The composition according to 4, wherein the lactic acid bacteria are Lactobacillus delbrueckii subspecies bulgaricus OLL1073R-1 (FERM BP-10741).
[6] The composition according to any one of 1 to 5, which is for ingestion by one selected from the group consisting of persons 65 years of age or older: young children; infants; neonates; persons suffering from any selected from the group consisting of chronic respiratory disease, cardiovascular disease, chronic renal disease, chronic liver disease, chronic hematologic disease, chronic metabolic disease, and neuromuscular disease: immunosuppressed persons; pregnant women; residents of long-term care facilities, persons with severe obesity; frail persons; persons receiving long-term aspirin administration; and cancer-bearing patients.
[7] The composition according to any one of 1 to 6, wherein the prevention of the secondary infectious disease after a viral infection, or the reduction of the risk of developing a secondary infectious disease after a viral infection is attained by suppression of CEACAM-1 expression.
[8] A composition for suppressing increase of expression of CEACAM-1, which contains an exopolysaccharide of lactic acid bacteria.
[9] The composition according to 8, which is for suppressing increase of expression of CEACAM-1 after a virus infection.
[10] The composition according to 9, wherein the virus is any selected from the group consisting of influenza virus, RS virus, parainfluenza virus, and hepatitis C virus.
[11] The composition according to any one of 8 to 10, wherein the lactic acid bacteria are those classified as Lactobacillus delbrueckii subspecies bulgaricus.
[12] The composition according to 11, wherein the lactic acid bacteria are Lactobacillus delbrueckii subspecies bulgaricus OLL1073R-1 (FERM BP-10741).
[13] The composition according to any one of 8 to 12, which contains the exopolysaccharide as fermented milk.
[14] An exopolysaccharide of lactic acid bacteria or a composition containing an exopolysaccharides of lactic acid bacteria, which is for use in a method for preventing, or for reducing the risk of developing a secondary infectious disease, especially a secondary bacterial infectious disease, after a virus infection; use of an exopolysaccharide of lactic acid bacteria in the manufacture of a composition for preventing, or for reducing the risk of developing a secondary infectious disease, especially a secondary bacterial infectious disease, after a virus infection; a method for preventing, or for reducing the risk of developing a secondary infectious disease, especially a secondary bacterial infectious disease, after a virus infection, which comprises the step of administering an exopolysaccharide of lactic acid bacteria or a composition containing an exopolysaccharide of lactic acid bacteria to a subject; or use of an exopolysaccharide of lactic acid bacteria or a composition containing an exopolysaccharide of lactic acid bacteria for preventing, or for reducing the risk of developing a secondary infectious disease, especially a secondary bacterial infectious disease, after a virus infection.
[15] The exopolysaccharide of lactic acid bacteria, composition, use, or method according to 14, wherein the virus is any of viruses that cause common cold syndrome.
[16] The exopolysaccharide of lactic acid bacteria, composition, use, or method according to 14 or 15, wherein the bacterial infectious disease is one caused by any bacterium selected from the group consisting of Staphylococcus aureus, Moraxella catarrhalis, Pseudomonas aeruginosa, Haemophilus influenzae, Klebsiella pneumoniae, Streptococcus pneumoniae, and glucose-nonfermentative gram-negative rods.
[17] The exopolysaccharide of lactic acid bacteria, composition, use, or method according to any one of 14 to 16, wherein the lactic acid bacterium is one classified as Lactobacillus delbrueckii subspecies bulgaricus.
[18] The exopolysaccharide of lactic acid bacteria, composition, use, or method according to 17, wherein the lactic acid bacterium is Lactobacillus delbrueckii subspecies bulgaricus OLL1073R-1 (FERM BP-10741).
[19] The exopolysaccharide of lactic acid bacteria, composition, use, or method according to any one of 14 to 18, wherein the composition is for ingestion by any selected from the group consisting of persons 65 years of age or older; young children; infants: neonates; persons suffering from any selected from the group consisting of chronic respiratory disease, cardiovascular disease, chronic renal disease, chronic liver disease, chronic hematologic disease, chronic metabolic disease, and neuromuscular disease; immunosuppressed persons; pregnant women: residents of long-term care facilities; persons with severe obesity; frail persons: persons receiving long-term aspirin administration; and cancer-bearing patients.
[20] The exopolysaccharide of lactic acid bacteria, composition, use, or method according to any one of 14 to 19, wherein the prevention of the secondary infectious disease, especially a secondary bacterial infectious disease, after viral infection or reduction of the risk of developing the same is attained by suppression of CEACAM-1 expression.
[21] An exopolysaccharide of lactic acid bacteria or a composition containing an exopolysaccharides of lactic acid bacteria, which is for use in a method for suppressing increase in expression of CEACAM-1 after a virus infection: use of an exopolysaccharide of lactic acid bacteria in the manufacture of a composition for suppressing increase in expression of CEACAM-1 after a virus infection: use of an exopolysaccharide of lactic acid bacteria or a composition containing an exopolysaccharide of lactic acid bacteria for suppressing increase in expression of CEACAM-1 after a virus infection; or a method for suppressing increase in expression of CEACAM-1 after a virus infection, which comprises the step of administering an exopolysaccharide of lactic acid bacteria or a composition containing an exopolysaccharide of lactic acid bacteria to a subject.
[22] The composition, use, or method according to 21, wherein the virus is any of viruses that cause common cold syndrome.
[23] The composition, use, or method according to 21 or 22, wherein the lactic acid bacterium is one classified as Lactobacillus delbrueckii subspecies bulgaricus.
[24] The composition, use, or method according to 23, wherein the lactic acid bacterium is Lactobacillus delbrueckii subspecies bulgaricus OLL1073R-1 (FERM BP-10741).
[25] The composition, use, or method according to any one of 21 to 24, wherein the exopolysaccharide is contained or used as fermented milk.

### Effect of the Invention

The composition of the present invention prevents secondary infectious diseases, especially a secondary bacterial infectious diseases, after viral infections or reduces the risk of developing the same.

According to the present invention, secondary infectious diseases, especially a secondary bacterial infectious diseases, after viral infections can be prevented or the risk of developing the same can be reduced by using exopolysaccharides of lactic acid bacteria, which have been eaten from very early years.

### BRIEF DESCRIPTION OF THE DRAWINGS

FIG. 1 The A549 cells were cultured for 1 hour in the presence of EPS and 1×10⁴ (A) or 1×10⁵ (B) pfu of influenza virus. The cells were washed with DMEM and further cultured for 6 hours in DMEM without EPS. The virus titer was significantly reduced (**p<0.01, *p<0.05) compared with that in the cells without EPS. These results are representative of two independent experiments.

FIG. 2 Effects of viral infection and EPS on the expression of mRNAs for adhesion molecules in A549 cells. The A549 cells were cultured for 1 hour in the presence of EPS and 1×10⁵ pfu of influenza virus. The cells were washed with DMEM and further incubated for 6 hours in DMEM without EPS. Expression levels of mRNAs were measured by quantitative RT PCR. The EPS treatment significantly decreased the expression amount of CEACAM-1, which was significantly increased by viral infection. Similar results were obtained in two independent experiments.

FIG. 3 Effects of viral infection and EPS on the number of adhered bacteria on A549 cells. The A549 cells were cultured for 1 hour in the presence of EPS and 1×10⁵ pfu of influenza virus. The cells were washed with DMEM without antibiotics and further incubated for 6 hours in DMEM without antibiotics. Then, 1×10⁵ cfu of S. aureus was added to each well and the cells were further cultured for 1 hour. After thorough washing, the A549 cells were removed from the plate, and then the number of the bacteria adhering to the cells was measured on agar plate. The number of the adhered bacteria tended to be decreased by the EPS treatment compared with that of the cells without the treatment. Similar results were obtained in four independent experiments.

### MODES FOR CARRYING OUT THE INVENTION

Hereafter, the present invention will be explained in detail.

The present invention relates to a composition containing an exopolysaccharide (EPS) of lactic acid bacteria. More precisely, the present invention relates to a composition for preventing a secondary infectious disease, especially a secondary bacterial infectious disease, after a viral infection or for reducing the risk of development of a secondary infectious disease, especially a secondary bacterial infectious disease, after a viral infection, which contains EPS of lactic acid bacteria as an active ingredient. In the following descriptions, the present invention may be explained for secondary bacterial infectious diseases as an example among secondary infectious diseases, but those skilled in the art can understand the present invention also for secondary infectious diseases caused by other pathogens by applying such explanations.

### [Active Ingredient]

The composition of the present invention contains EPS of lactic acid bacteria as an active ingredient. The term lactic acid bacterium is a general term for microorganisms that utilize glucose to produce lactic acid at a yield of 50% or higher based on the saccharide, and have such characteristics as being gram-positive cocci or bacilli (rods), showing no motility, usually showing no spore-forming ability (there are also lactic acid bacteria having a spore-forming ability such as Bacillus coagulans), and being catalase negative, as physiological properties. Lactic acid bacteria have been consumed since ancient times in many parts of the world through fermented milk and other products, and can be said to be extremely safe microorganisms. Lactic acid bacteria are classified into multiple genera. The EPS of lactic acid bacteria contained in the composition of the present invention is preferably produced by a Lactobacillus lactic acid bacterium classified in the genus Lactobacillus.

The EPS used in the composition of the present invention is not particularly limited so long as it provides the desired effect. As for structure, EPS produced by lactic acid bacteria can be classified into those that are homopolysaccharides and those that are heteropolysaccharides (e.g., those composed of galactose and glucose), and may be modified by phosphorylation and sulfation, but all can be used as the active ingredient of the composition of the present invention. One class of preferred examples of EPS are those containing at least one of neutral polysaccharide and acidic polysaccharide consisting of neutral polysaccharide to which phosphate group is added. It is known that such EPS are produced by Lactobacillus delbrueckii subsp. bulgaricus, Lactococcus lactis subsp. cremoris, and so forth. The EPS used in the present invention may consist of one type of EPS or a combination of two or more types of EPS.

Examples of particularly preferred EPS-producing lactic acid bacterium used in the composition of the present invention include lactic acid bacteria of the genus Lactobacillus. Examples of lactic acid bacterium of the genus Lactobacillus include species bulgaricus, casei, acidophilus, plantarum, and so forth. The term “lactic acid bacterium belonging to the genus Lactobacillus” is used herein to refer to a lactic acid bacterium belonging to any of 25 genera newly established by the reorganization of lactic acid bacteria published in the article “A taxonomic note on the genus Lactobacillus: description of 23 novel genera, emended description of the genus Lactobacillus Beijerinck 1901, and union of Lactobacillaceae of Lactobacillaceae and Leuconostocaceae” contained in INTERNATIONAL JOURNAL OF SYSTEMATIC AND EVOLUTIONARY MICROBIOLOGY, Volume 70, Issue 4 published on Apr. 15, 2020, namely, genera Lactobacillus, Paralactobacillus, Holzapfelia, Amylolactobacillus, Bombilactobacillus, Companilactobacillus, L. apidilactohacillus, Agrilactobacillus, Schleiferilactobacillus, Loigolactobacillus, Lacticaseibacillus, Latilactobacillus, Dellaglioa, Liyuorilactobacillus, Ligilacto-bacillus, Lactiplantibacillus, Furfhrilactohacillus, Paucilactohacillus, Limosilactobacillus, Fructilactobacillus, Acetilactobacillus, Apilactobacillus, Levilactobacillus, Secundilactobacillus, and Lentilactobacillus. Among these lactic acid bacteria of the genus Lactobacillus, lactic acid bacteria classified as the species bulgaricus (also called bulgaricus bacteria) are preferred for the present invention. Further, among the lactic acid bacteria of the genus Lactobacillus, those classified as Lactobacillus delbrueckii subspecies bulgaricus Lactobacillus delbrueckii subsp. bulgaricus) are more preferred. In other words, one particularly preferred example of EPS used in the composition of the present invention is EPS of lactic acid bacteria classified as Lactobacillus delbrueckii subspecies bulgaricus.

In a particularly preferred embodiment, the lactic acid bacterium is Lactobacillus delbrueckii subspecies bulgaricus OLL1073R-1 (accession number, FERM BP-10741; also referred to as “bulgaricus R-1 strain”. In other words, one particularly preferred example of EPS used in the composition of the present invention is EPS of the bulgaricus R-1 strain.

The bulgaricus R-1 strain was deposited as an international deposition at the independent administrative agency, National Institute of Technology and Evaluation, International Patent Organism Depositary (IPOD, NITE, Room 120, 2-5-8 Kazusakmadzhi, Kisarazu, Chiba, Japan) under the terms of the Budapest Treaty. (Depositor, Meiji Co., Ltd.; date of deposition, Nov. 29, 2006; accession number, FERM BP-10741).

The EPS of lactic acid bacteria contained in the composition of the present invention may be included as a lactic acid bacterium fermentation product. Lactic acid bacterium fermentation products include fermented products produced by lactobacilli themselves as well as processed products thereof. The lactic acid bacterium fermentation products themselves include, for example, fermented milk (specifically, yogurt, etc.). The processed products include, for example, roughly purified products, culture filtrates and culture supernatants obtained from fermented products by sterilization thorough filtration, centrifugation, or membrane separation, concentrated products produced by concentrating culture filtrates or culture supernatants, and dried products of the concentrated products.

Conventional techniques can be used for the preparation of EPS of a lactic acid bacterium, and if more detailed conditions are required, the examples mentioned in this description and so forth can be referred to. When EPS of a lactic acid bacterium is prepared as a lactic acid bacterium fermentation product, fermented milk containing EPS can be produced by adding lactic acid bacteria that produces EPS to raw milk as a starter, and allowing fermentation to make the bacterium produce EPS in the fermented product. The conditions for fermentation, such as specification of raw milk, fermentation temperature, and fermentation time, are not particularly limited so long as the lactic acid bacterium used can produce EPS, and those skilled in the art can appropriately chose them.

### [Use]

The composition of the present invention containing EPS of lactic acid bacteria can be used to prevent a secondary bacterial infectious disease after a viral infection or reduce the risk of developing a secondary bacterial infectious disease after a viral infection. The secondary bacterial infectious disease after a viral infection refers to a bacterial infectious disease caused after a viral infection by bacteria infecting the lungs, bronchi or the like that have been damaged by the viral infection. Prevention of a secondary bacterial infectious disease after a viral infection or reduction of the risk of developing the same include prevention and reduction of the risk of secondary bacterial infections that do not lead to development of symptoms thereof, after a viral infection.

The composition of the present invention is effective against viruses causing common cold syndrome, viruses causing gastrointestinal tract infectious diseases, viruses causing rash infectious diseases, viruses causing liver infectious diseases, viruses causing nervous system infectious diseases, and so forth, and is particularly effective against viruses causing common cold syndrome. Viruses causing common cold syndrome include influenza viruses, rhinoviruses, coronaviruses, RS viruses, parainfluenza viruses, and adenoviruses.

The present invention is particularly effective against secondary bacterial infectious diseases after infections caused by viruses that are transmitted via cell-side receptor molecules, of which infection increases expression of carcinoembryonic antigen-related cell adhesion molecule 1 (CEACAM-1), such as influenza viruses. Since infection of bronchial epithelial cells with RS viruses or human parainfluenza virus 3 also increases CEACAM-1 expression and pneumococcal adhesion (Avadhanula V, Rodriguez C A, DeVincenzo J P, Wang Y, Webby R J, Ulett G C, and Adderson E E, Respiratory Viruses Augment the Adhesion of Bacterial Pathogens to Respiratory Epithelium in a Viral Species- and Cell Type-Dependent Manner, J Virol, 80, 1629-1636, 2006), EPS of lactic acid bacteria is considered to be also effective in suppressing not only secondary bacterial infectious diseases caused by influenza virus infection but also secondary bacterial infectious diseases caused by RS virus and parainfluenza virus infection. Among the viruses that cause liver infectious diseases, hepatitis C virus (HCV) uses CEACAM-1 as a receptor. Therefore, EPS of lactic acid bacteria is considered to be also effective in suppressing secondary bacterial infectious diseases caused by infection with viruses that cause liver infectious diseases, such as hepatitis C virus (HCV).

Influenza viruses are minus single-stranded RNA viruses of the family Orthomyxoviridae and have envelopes. Viruses of the family Orthomyxoviridae include influenza A viruses, influenza B viruses, influenza C viruses, Thogoto viruses, and isaviruses.

Pathogens of secondary infectious diseases include pathogens of which adhesion to epithelial cells is promoted by adhesion molecules induced by viruses, such as bacteria, viruses, fungi, and parasites. Pathogens that cause secondary bacterial infectious diseases include Staphylococcus aureus, Moraxella catarrhalis, Pseudomonas aeruginosa, Haemophilus influenzae, Klebsiella pneumoniae, Streptococcus pneumoniae, glucose-nonfermenting gram-negative rods, Escherichia coli, and Neisseria gonorrhoeae. The term glucose-nonfermenting gram-negative rod is a general term for gram-negative rods that do not anaerobically ferment glucose, and they are found in soil and aqueous environments as well as on human skin and mucous membranes. They have low nutrient requirements, and can grow and survive for long periods of time even in nutrient-poor wet environments. Pseudomonas spp., Burkholderia spp., Acinetobacter spp, Stenotrophomonas spp., Chryseobacterium spp., Achromobacter spp. and so forth are frequently detected in clinical specimens and are considered opportunistic infectious bacteria to watch for in hospitals. Glucose-nonfermenting gram-negative rods include multidrug-resistant Pseudomonas aeruginosa (MDRP) and multidrug-resistant Acinetobacter bacteria (MDRA). Examples of MDRA include Acinetobacter baumannii, Acinetobacter genomic species 13TU, and other Acinetobacter genomic species. Other pathogens that can cause other secondary infectious diseases include the fungus Candida albicans.

The prevention or reduction of the risk of the development includes suppressing, inhibiting, or reducing development or manifestation of the objective disease or condition, and reducing the risk of development or manifestation thereof. The prevention or reduction of the risk of development includes medical treatments performed by physicians and nurses, midwives, and others under the direction of physicians, and non-therapeutic actions performed by persons other than physicians, such as pharmacists, nutritionists (including registered dietitians and sports nutritionists), health workers, midwives, nurses, clinical technicians, sports instructors, drug manufacturers, drug sellers, food manufacturers, and food sellers. In addition, the prevention or reduction of the risk of development includes recommendations for intake of certain foods, nutritional guidance (including guidance on nutrition necessary for medical treatment of injured or sick persons and guidance on nutrition for the maintenance and promotion of good health).

The composition of the present invention can also be used to suppress expression of the carcinoembryonic antigen-related cell adhesion molecule 1 (CEACAM-1).

In one of the preferred embodiments of the present invention, prophylactic agents for influenza infection are excluded, and methods for treating influenza infectious diseases are excluded.

### Composition

### (Food Composition Etc.)

The composition of the present invention can be in the form of a food composition or pharmaceutical composition. Food and drug are not limited to those for humans, and may be those for animals other than human, unless especially indicated. The food may be a common food, functional food, or nutritional composition, or a therapeutic diet (diet for the purpose of treatment, for which a medical practitioner writes a dietary prescription, and which is cooked by a dietitian or the like according to the prescription), dietetic food, ingredient-modified food, care food, or treatment-supporting food, unless especially indicated. The food is not limited to a solid food, but it may be a food in the form of liquid, for example, drink, drinkable preparation, liquid food, or soup, unless especially indicated. Functional food refers to a food that can give a predetermined functionality to a living body, and includes health foods at large, such as foods for specified health uses (abbreviated as “Tokuho” in Japanese, including conditional foods for specified health use), foods with function claims, foods with health claims including foods with nutrient function claims, foods for special dietary uses, supplements (for example, those of various kinds of dosage forms such as tablet, coated tablet, sugar-coated tablet, capsule and solution), and cosmetic food (for example, diet foods). In the present invention, the “functional foods” include health foods to which the health claim based on the food standards of CODEX (JOINT FAO/WHO FOOD STANDARDS PROGRAMME CODEX ALIMENTARIUS COMMISSION) is applied.

### (Subject)

The composition of the present invention is suitable to be ingested by or administered to subjects for whom it is desirable to treat infections caused by viruses, for example, subjects for whom it is desirable to avoid secondary infectious diseases, especially a secondary bacterial infectious diseases, after viral infection. Such subjects include: persons 65 years of age or older; young children (1 to 6 years of age); infants (younger than 1 year of age); neonates (within 28 days of birth); persons suffering from any selected from the group consisting of chronic respiratory disease, cardiovascular disease, chronic renal disease, chronic liver disease, chronic hematologic disease, chronic metabolic disease, and neuromuscular disease; immunosuppressed persons; pregnant women; residents of long-term care facilities; persons with severe obesity; frail persons; persons receiving long-term aspirin administration; and cancer-bearing patients.

### (Administration Route Etc.)

The composition of the present invention may be administered orally, parenterally, e.g., via a tube (gastrostomy or enterostomy), or intranasally, but oral administration is preferred.

The composition of the present invention is preferably used before or immediately after viral infection. This is because the active ingredient. EPS, is expected to be effective through a mechanism that inhibits the adhesion of secondary bacterial infectious disease-causing bacteria by suppression of increased expression of CEACAM-1 occurring due to viral infection. The composition of the present invention is expected to be highly effective when ingested on a daily basis, or in advance before infection when the risk of viral infection is high, or immediately when infection may have occurred. Continuous ingestion is preferred.

Non-patent document 9 mentioned above reports that an active ingredient is administered prior to influenza virus infection to enhance immunity prior to infection, and thereby suppress influenza virus infection. However, this reference does not describe nor suggest secondary bacterial infectious diseases that may occur after viral infection, and it cannot be not understood that secondary bacterial infectious diseases were suppressed inherently in the reference.

### (Content and Dose of Active Ingredient)

The content of EPS of lactic acid bacteria in the composition of the present invention should be such an amount that the desired effect is exhibited. Although the dose or ingestion amount of the composition may be appropriately set by taking various factors such as age, weight, and symptoms of the subject into consideration, the daily dose of EPS of lactic acid bacteria can be, for example, 0.1 mg or more, and it is preferably 0.6 mg or more, more preferably 1 mg or more, especially preferably 3 mg or more. No matter how the minimum daily dose of EPS is defined, the maximum daily dose of EPS can be 500 mg or less, and it is preferably 300 mg or less, especially preferably 250 mg or less.

The amount of EPS of lactic acid bacterium per administration or per meal, i.e., dose for 1 time, can be, for example, 0.03 mg or more, and it is preferably 0.2 mg or more, more preferably 1 mg or more. No matter how the minimum amount of EPS for 1 time is defined, the maximum amount of EPS for 1 time can be 200 mg or less, and it is preferably 100 mg or less, more preferably 70 mg or less, especially preferably 30 mg or less.

When EPS of lactic acid bacteria contained in the composition of the present invention is used as a composition such as fermented milk, the daily amount as the composition can be, for example, 30 g or more, and it is preferably 50 g or more, more preferably 60 g or more, especially preferably 100 g or more. No matter how the minimum daily amount as fermented milk is defined, the maximum daily amount as fermented milk can be, for example, 1500 g or less, and it is preferably 1200 g or less, more preferably 900 g or less, still more preferably 600 g or less,

The amount of the composition for 1 time can be, for example, 10 g or more, and it is preferably 20 g or more, more preferably 30 g or more. No matter how the minimum amount of the composition for 1 time is defined, the maximum amount of the composition for 1 time can be, for example, 500 g or less, and it is preferably 400 g or less, more preferably 200 g or less, especially preferably 125 g or less,

The composition may be administered or ingested once a day, or multiple times a day, e.g., it may be administered or ingested at each meal, i.e., three times a day. The composition contains EPS of lactic acid bacteria, which have been eaten from very early years, as the active ingredient. Thus, the composition of the present invention is suitable for long-term ingestion, because the active ingredient thereof is EPS of lactic acid bacteria, which have been eaten from very early years. Therefore, the composition may be ingested repeatedly or over a long period of time, and it may be administered or ingested continuously for, for example, 3 days or longer, preferably 1 week or longer, more preferably 4 weeks or longer, especially preferably 1 month or longer.

### (Other Ingredients and Additives)

The composition of the present invention may contain another active ingredient or nutritional ingredient acceptable for foods or drugs. Examples of such an ingredient include amino acids (for example, lysine, arginine, glycine, alanine, glutamic acid, leucine, isoleucine, and valine), saccharides (glucose, sucrose, fructose, maltose, trehalose, erythritol, maltitol, paratinose, xylitol, and dextrin), electrolytes (for example, sodium, potassium, calcium, magnesium salts etc.), vitamins (for example, vitamin A, vitamin B1, vitamin B2, vitamin B6, vitamin B12, vitamin C, vitamin D, vitamin E, vitamin K, biotin, folic acid, pantothenic acid, and nicotinic acid), minerals (for example, copper, zinc, iron, cobalt, and manganese), antibiotics, dietary fibers, proteins, lipids, and so forth.

The composition may also further contain an additive acceptable for foods or drugs. Examples of such an additive include inactive carriers (solid and liquid carriers), excipients, surfactants, binders, disintegrating agents, lubricants, dissolving aids, suspending agents, coating agents, colorants, preservatives, buffering agents, pH adjustors, emulsifiers, stabilizers, sweeteners, antioxidants, perfumes, acidulants, and natural substances. More specific examples include water, other aqueous solvents, pharmaceutically acceptable organic solvents, collagen, polyvinyl alcohol, polyvinylpyrrolidone, carboxyvinyl polymer, sodium alginate, water-soluble dextran, water-soluble dextrin, carboxymethyl starch sodium, pectin, xanthan gum, gum arabic, casein, gelatin, agar, glycerin, propylene glycol, polyethylene glycol, vaseline, paraffin, stearyl alcohol, stearic acid, human serum albumin, mannitol, sorbitol, lactose, sucralose, stevia, aspartame, acesulfame potassium, citric acid, lactic acid, malic acid, tartaric acid, phosphoric acid, acetic acid, fruit juice, vegetable juice, and so forth.

### (Dosage Form or Shape)

The composition of the present invention in the form of a food may be prepared in an arbitrary form such as solid, liquid, mixture, suspension, powder, granule, paste, jelly, gel, and capsule. The composition of the present invention in the form of a food can be made in such an arbitrary form as dairy product, supplement, confectionery, drink, drinkable preparation, seasoning, processed food, daily dish, and soup. More specifically, the composition of the present invention may be in the form of fermented milk, lactic acid bacterium beverage, lactic beverage, milk beverage, soft drink, ice cream, tablet, cheese, bread, biscuit, cracker, pizza crust, special formula powdered milk, liquid food, food for sick persons, nutritional food, frozen food, processed food, or the like, and can also be in the form of granule, powder, paste, thick solution, etc. to be mixed with beverages and foods for ingestion. Fermented milk means fermented milk and lactic acid beverages as defined in the “Ministerial Ordinance on Milk and Milk products Concerning Compositional Standards etc.” issued by Ministry of Health and Welfare, Japan (hereinafter referred to as the “Ministerial Ordinance on Milk, etc.”). Fermented milk defined in the Ministerial Ordinance on Milk, etc. is a product prepared by fermenting milk or milk product having a nonfat milk solid content not lower than that of milk with lactic acid bacteria or yeast and making it into paste or liquid, or a product obtained by freezing the foregoing.

One of the preferred embodiments of the composition in the form of food is fermented milk obtained by fermenting raw material milk with an EPS-producing lactic acid bacterium as a starter. The fermented milk can contain microorganisms such as yeast other than the objective lactic acid bacterium. In one preferred embodiment, the fermented milk contains one or more types of lactic acid bacteria, but may or may not contain other microorganisms, such as yeast. The raw material milk includes milk of animal origin and processed products thereof, such as cow's milk, skimmed milk, skimmed milk powder, skimmed milk concentrate, filtered concentrate or permeate of milk, condensed milk, whey, milk protein concentrate (MPC), whey protein concentrate (WPC), buttermilk and fresh cream. The raw material milk may or may not contain vegetable milk, e.g., soymilk, almond milk, oat milk, coconut milk, rice milk, and hemp milk.

The composition of the present invention as a pharmaceutical can be made into any dosage form suitable for oral administration, including solid dosage forms such as tablet, granule, powder, pill, and capsule, liquid dosage forms such as solution, suspension, and syrup, gel, aerosol, and so forth.

### (Others)

In the manufacture of the composition of the present invention, time of adding EPS of lactic acid bacteria can be appropriately chosen. Unless the characteristics of EPS of lactic acid bacteria are not markedly degraded, the time of the addition is not particularly limited. For example, it can be added by mixing a culture containing EPS obtained by cultivating an EPS-producing lactic acid bacterium or a roughly purified or purified product thereof with the raw material. Alternatively, when the composition of the present invention is implemented as fermented milk, fermented milk containing EPS can be produced by adding EPS of lactic acid bacteria by mixing a culture containing EPS or a roughly purified or purified product thereof with the raw material or fermented milk after the fermentation, or adding an EPS-producing bacterium as a starter to raw material milk, and allowing fermentation so that EPS is produced.

The composition of the present invention can have an indication describing the purpose of use (intended use) and can also have an indication describing a recommendation for ingestion thereof by specific subjects.

The composition of the present invention can have an indication describing that the composition can be used for preventing secondary bacterial infectious diseases after viral infection, or for reducing the risk of developing the same, or an indication describing a recommendation for ingestion thereof by specific subjects. The indication may be a direct or indirect indication. Examples of the direct indication include descriptions on tangible articles such as the product itself, package, container, label, and tag, and examples of the indirect indication includes advertising and campaign activities using such places or means as web site, shop, pamphlet, exhibition, seminar such as media seminar, book, newspaper, magazine, television, radio, postal matter, E-mail, and sound.

The present invention provides a method for preventing a secondary bacterial infectious disease after a viral infection, or for reducing the risk of developing the same, comprising the step of administering an exopolysaccharide of lactic acid bacteria, and such a method may comprise the step of testing the subject for the presence or absence of secondary bacterial infection after the step of administering the active ingredient. Such test may be physical examination, medical interview, antigen test, antibody test, and PCR test. The test may be performed by the subject himself or herself, or by someone other than the subject.

Hereafter, the present invention will be more specifically explained with reference to examples. However, the technical scope of the present invention is not limited by these examples.

### EXAMPLES

### Materials and Methods

### (Cells, Viruses, Bacteria, and EPS)

A549 cells (human alveolar basal epithelial adenocarcinoma cells) were purchased from the RIKEN BioResource Center Cell Bank (Tsukuba, Japan). The cells were cultured in DMEM containing 10% FBS, 100 U/ml penicillin, 100 μg/ml streptomycin (Sigma, MO, USA) and MEM non-essential amino acids (Thermo Fisher Scientific, MA, USA) in 5% CO₂ at 37° C. Influenza virus A/Puerto Rico/8/34 (H1N1) was kindly provided by the University of Tokyo.

S. aureus strain ATCC 29213 was purchased from the American Type Culture Collection (VA, USA). S. aureus cells were cultured in brain heart infusion broth (Becton Dickinson. MD, USA) at 37° C. S. aureus cells in growth phase were washed 3 times with PBS, and the bacterial cell number was calculated by using a standard curve based on absorbance of 600 nm.

EPS produced by Lactobacillus delbrueckii subsp. bulgaricus OLL1073 R-1 was obtained by culturing Lactobacillus delbrueckii subsp. bulgaricus OLL1073R-1 in 10 mass % nonfat milk powder medium, and purifying EPS in the obtained culture. That is, trichloroacetic acid was added to the culture after incubation at 37° C. for 18 hours at a final concentration of 10 mass % to remove denatured proteins, cold ethanol was added, and the mixture was allowed to stand at 4° C. for 2 hours to obtain precipitates containing EPS. The precipitates were then dialyzed against MilliQ water using a dialysis membrane (molecular weight cut off 6,000 to 8,000), nucleic acids and proteins were enzymatically decomposed, and ethanol precipitation was performed again to obtain precipitates. The precipitates were dissolved in MilliQ water, and after dialysis was performed again, EPS was lyophilized to be purified. The lyophilized product was dissolved in distilled water sterilized by autoclaving. The EPS solution was filtered through a 0.22 μm syringe filter, and the filtered solution was frozen at −80° C. until use.

### (Influenza Virus Infection in A549 Cells)

Culture of the A549 cells was started at a density of 1×10⁵ cells/200 μl/well on a 96-well flat bottom plate on day −1. On day 0, the culture medium of the A549 cells was changed to fresh medium, and two-fold serially diluted EPS solutions were added to each well. Simultaneously, the A549 cells were infected with 1×10⁴ or 1×10⁵ pfu (per well) of influenza virus, and cultured for 1 hour in 5% CO₂ at 37° C. The cells were washed 3 times with DMEM and further incubated in DMEM without EPS for 6 hours in 5% CO₂ at 37° C. In some experiments, 250 nM baloxavir acid (BXA, Shionogi CO., Ltd., Osaka, Japan) was added to the wells during the experiments as a positive control of virus replication inhibitor. After incubation for 6 hours, RNAs were extracted from each infected A549 cells.

### (Purification of RNA and Quantitative RT-PCR)

Purification of RNAs from the infected A549 cells and cDNA synthesis were performed by using Power SYBR (registered trademark) Green Cell-to-CT™ kit according to the manufacturer's instructions (Thermo Fisher Scientific). The influenza virus M gene region was amplified with primers for quantitative RT-PCR, 5′-GGCAAATGGTACAGGCAATG-3′ (SEQ ID NO: 1) and 5′-AGCAACGAGAGGATCACTTG-3′ (SEQ ID NO: 2) (Moradi et al., 2017). cDNA was prepared on the basis of the influenza virus RNA of known virus titer by the 50% tissue culture infective dose (TCID₅₀) method, and used as a standard for quantitative RT-PCR. The primers for quantitative RT-PCR of tight junction molecule genes or adhesion molecule genes of the A549 cells used in this study were indicated in the following table.

TABLE 1
Sequences of primer for quantitative RT-PCR

| | | SEQ | | SEQ |
|---|---|---|---|---|
| | | ID | | ID |
| Gene name | Forward Primer (5′-3′) | NO.: | Reverse Primer (5′-3′) | NO.: |
| ZO-1 | TGGTGTCCTACCTAATTCAACTCA | 3 | CGCCAGCTACAAATATTCCAACA | 4 |
| Occludin | ACAGAGCAAGATCACTATGAGACA | 5 | TGTTGATCTGAAGTGATAGGTGGA | 6 |
| Claudin-1 | CCAGTCAATGCCAGGTACGAAT | 7 | TTGGTGTTGGGTAAGAGGTTGTT | 8 |
| E-cadherin | ATAGAGAACGCATTGCCACATACA | 9 | TTCTGATCGGTTACCGTGATCA | 10 |
| ICAM-1 | GCCACTGGGCAAGAACCT | 11 | TCAGTGCGGCACGAGAAA | 12 |
| VCAM-1 | TGAACCCAAACAGAGGCAGAGT | 13 | GGTATCCCATCACTTGAGCCAGG | 14 |
| CEACAM-1 | GCTGGCATTGTGATTGGAGTA | 15 | TTAGGTGGGTCATTGGAGTG | 16 |
| Fibronectin | TAGCCCTGTCCAGGAGTTCA | 17 | CTGCAAGCCTTCAATAGTGT | 18 |
| PAF-r | GCAATTGCCAGGCCACAAC | 19 | AAGGGTCACCTGGTCATGGAG | 20 |
| E-selectin | AGCAGAGTTTCACGTTGCAGG | 21 | TGGCGCAGATAAGGCTTCA | 22 |
| β-actin | AGAAAATCTGGCACCACACC | 23 | AGAGGCGTACAGGGATAGCA | 24 |

The quantitative RT-PCR was performed by using LightCycler 480 Probe Master and LightCycler 480 instrument with the included software program (Roche Diagnostics, Mannheim, Germany). In some experiments, each sample was calibrated to the internal standard (β-ac tin) level and normalized to the average value of the control samples.

(Measurement of S. aureus Adhesion to Cells)

Culture of the A549 cells was started at a density of 1×10⁵ cells/200 μl/well on a 96-well flat bottom plate on day −1. On day 0, the culture medium of the A549 cells was changed to fresh medium, and 400 μg EPS or 250 nM BXA was added to each well. Simultaneously, the A549 cells were infected with 1×10⁵ pfu of influenza virus, and cultured for 1 hour in 5% CO₂ at 37° C. The cells were washed 3 times with DMEM without antibiotics and further incubated in DMEM without EPS and antibiotics or DMEM without antibiotics but with 250 nM BXA for 6 hours in 5% CO₂ at 37° C. After incubation for 6 hours, 1×10⁵ cfu of S. aureus was added to each well, and the cells were incubated for another 1 hour. The cells were thoroughly washed 10 times with PBS to remove unattached bacteria. The A549 cells were detached from the plate bottom with TrypLeX™ Express (Thermo Fisher Scientific) and numbers of the attached bacteria on the A549 cells were counted on a brain heart infusion agar plate (Becton Dickinson) after the cultivation.

### (Statistical Analysis)

Statistical analyses were performed by using the Microsoft Excel software program (Microsoft, WA, USA). The statistical significance of the findings was calculated by using the unpaired t-test for all the experimental analyses. When P-values were less than 0.05, it was determined that there was statistical significance. All the values are presented as mean±standard deviation.

### Results

### (When EPS Treatment is Done Before Infection, EPS Inhibits Influenza Virus Infection)

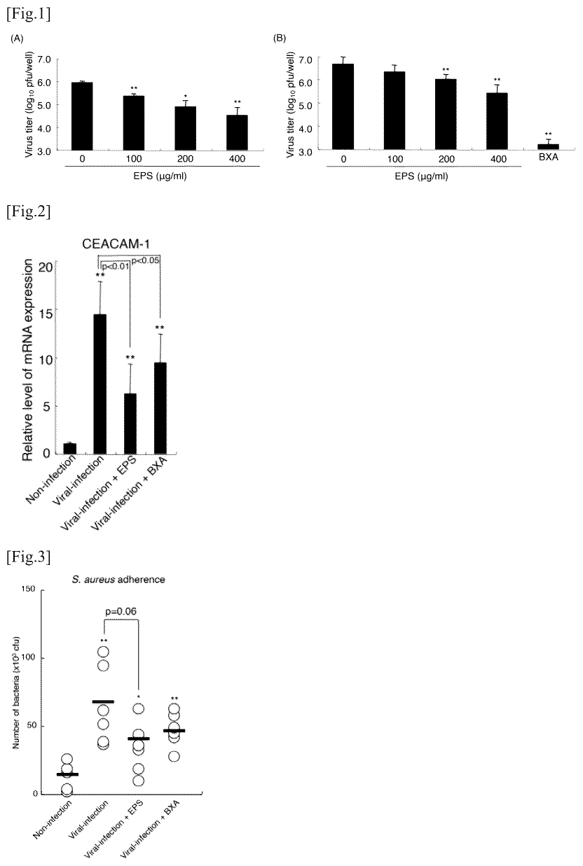

To investigate whether EPS is effective against influenza virus infection, A549 cells were treated with various concentrations of EPS before or after virus infection. When the A549 cells were treated with EPS before virus infection at 1×10⁴ (FIG. 1A) or 1×10⁵ pfu (FIG. 1B), EPS significantly reduced influenza virus titer in the infected A549 cells in a dose-dependent manner at EPS doses of 100, 200, and 400 μg/ml or 200 and 400 μg/ml, compared with the untreated group. The results suggest that EPS inhibits the adhesion and invasion of influenza viruses to A549 cells.

As it had been reported that infection and replication of influenza virus on airway epithelial cells and pulmonary alveolus cause serious damage to the barrier function including tight junction molecules (Non-patent document 4 mentioned above). mRNA expressions of tight junction molecules after the viral infection were evaluated in the EPS pre-treatment study. However, no difference in ZO-1, occludin, claudin-1 and E-cadherin mRNA expressions was found between the non-viral infection group and viral-infection group irrespective of whether the infected A549 cells were treated with EPS or BXA or not. These data suggest that the disruptions of tight junction are not caused by influenza virus infection under these experimental conditions. In addition, EPS did not have any effects on expressions of mRNAs for tight junction molecules.

(Expression of CEACAM-1 mRNA was Increased by Influenza Virus Infection and the Increased Expression of CEACAM-1 mRNA was Reduced by EPS Treatment)

As it had been reported that respiratory viruses promote bacterial adhesion to epithelial cells through adhesion molecules induced by viruses (Non-patent document 8 mentioned above), expressions of mRNAs for adhesion molecules after the viral infection were determined in the EPS pre-treatment study. As shown in FIG. 2, CEACAM-1 mRNA expression of the virus-infected A549 cells was significantly over 10-fold higher than that of the non-infected A549 cells.

When A549 cells were treated with EPS on viral infection, the increased CEACAM-1 mRNA expression caused by influenza virus infection was markedly reduced as shown in FIG. 2. CEACAM-1 mRNA expression of the virus-infected A549 cells treated with BXA was significantly reduced compared with that of the virus-infected A549 cells. On the other hand, ICAM-1, VCAM-1, fibronectin, PAF-r and E-selectin mRNA expressions were not changed irrespective of whether the infected A549 cells were treated with EPS or BXA or not.

### (Numbers of Adhered Bacteria to Virus-Infected A549 Tended to be Decreased by EPS Treatment)

Given that CEACAM-1 mRNA expression in A549 cells increased during influenza virus infection and the increased CEACAM mRNA expression in virus-infected A549 was reduced by EPS treatment on viral infection, secondary bacterial adherence assays were performed according to the methods previously described in Non-patent document 8 mentioned above. As shown in FIG. 3, numbers of adhered bacteria on the virus-infected A549 cells significantly increased compared with those on the non-infected A549 cells.

The increased numbers of adhered bacteria caused by influenza virus infection tended to be reduced by EPS treatment before viral infection. Number of adhered bacteria on virus-infected A549 cells treated with BXA did not decrease compared with the number of adhered bacteria on the virus-infected A549 cells.

### [Production of Fermented Dairy Product (Plain Yogurt)]

By mixing milk, dairy product (derived from milk) and water so that the final product has a nonfat milk solid content of 9.5% and a milk fat content of 3.0%, yogurt base mix is prepared. Then, the prepared yogurt base mix is homogenized, heat sterilized at 95° C. for 5 minutes, and then cooled to approximately 40° C. Lactobacillus delbrueckii subsp. bulgaricus OLL1073R-1 and a lactic acid bacterium strain belonging to Streptococcus thermophilus are added to the sterilized yogurt base mix as a starter, and fermentation is allowed to produce fermented milk. The produced fermented milk can be used to prevent secondary bacterial infectious diseases after viral infection or reduce the risk of developing secondary bacterial infectious diseases after viral infection.

### [Sequences Listed in the Sequence Listing]

SEQ ID NO: 1, Influenza virus M gene region RT-PCR forward primer
SEQ ID NO: 2, Influenza virus M gene region RT-PCR reverse primer
SEQ ID NO: 3, ZO-1 RT-PCR forward primer
SEQ ID NO: 4, ZO-1 RT-PCR reverse primer
SEQ ID NO: 5, Occludin RT-PCR forward primer
SEQ ID NO: 6, Occludin RT-PCR reverse primer
SEQ ID NO: 7, Claudin-1 RT-PCR forward primer
SEQ ID NO: 8, Claudin-1 RT-PCR reverse primer
SEQ ID NO: 9, E-Cadherin RT-PCR forward primer
SEQ ID NO: 10, E-Cadherin RT-PCR reverse primer
SEQ ID NO: 11, ICAM-1 RT-PCR forward primer
SEQ ID NO: 12, ICAM-1 RT-PCR reverse primer
SEQ ID NO: 13, VCAM-1 RT-PCR forward primer
SEQ ID NO: 14, VCAM-1 RT-PCR reverse primer
SEQ ID NO: 15, CEACAM-1 RT-PCR forward primer
SEQ ID NO: 16, CEACAM-1 RT-PCR reverse primer
SEQ ID NO: 17, Fibronectin RT-PCR forward primer
SEQ ID NO: 18, Fibronectin RT-PCR reverse primer
SEQ ID NO: 19, PAF-r RT-PCR forward primer
SEQ ID NO: 20, PAF-r RT-PCR reverse primer
SEQ ID NO: 21, E-Selectin RT-PCR forward primer
SEQ ID NO: 22, E-Selectin RT-PCR reverse primer
SEQ ID NO: 23, β-Actin RT-PCR forward primer
SEQ ID NO: 24, β-Actin RT-PCR reverse primer

1. A method for preventing a secondary infectious disease after a virus infection, or for reducing risk of developing a secondary infectious disease after a virus infection, the method comprising:
administering a composition comprising an exopolysaccharide of lactic acid bacteria to a subject in need of preventing a secondary infectious disease after a virus infection, or of reducing risk of developing a secondary infectious disease after a virus infection.

2. The method according to claim 1, wherein the virus is any of viruses that cause common cold syndrome.

3. The method according to claim 1, wherein the infectious disease is one caused by any selected from the group consisting of Staphylococcus aureus, Moraxella catarrhalis, Pseudomonas aeruginosa, Haemophilus influenzae, Klebsiella pneumoniae, Streptococcus pneumoniae, and glucose-nonfermentative gram-negative rods.

4. The method according to claim 1, wherein the lactic acid bacteria are those classified as Lactobacillus delbrueckii subspecies bulgaricus.

5. The method according to claim 4, wherein the lactic acid bacteria are Lactobacillus delbrueckii subspecies bulgaricus OLL1073R-1 (FERM BP-10741).

6. The method according to claim 1, wherein the subject is any selected from the group consisting of persons 65 years of age or older; young children; infants; neonates; persons suffering from any selected from the group consisting of chronic respiratory disease, cardiovascular disease, chronic renal disease, chronic liver disease, chronic hematologic disease, chronic metabolic disease, and neuromuscular disease; immunosuppressed persons; pregnant women; residents of long-term care facilities; persons with severe obesity; frail persons; persons receiving long-term aspirin administration; and cancer-bearing patients.

7. The method according to claim 1, wherein the method comprises suppressing carcinoembryonic antigen-related cell adhesion molecule 1 (CEACAM-1) expression, and thereby the prevention of a secondary infectious disease after a viral infection, or reduction of the risk of developing a secondary infectious disease after a viral infection is attained.

8. A method for suppressing increase of expression of CEACAM-1, the method comprising:
administering a composition comprising an exopolysaccharide of lactic acid bacteria to a subject in need of suppressing increase of expression of CEACAM-1.

9. The method according to claim 8, the method comprising suppressing increase of expression of CEACAM-1 after a virus infection.

10. The method according to claim 9, wherein the virus is any selected from the group consisting of influenza virus, RS virus, parainfluenza virus 3, and hepatitis C virus.

11. The method according to claim 8, wherein the lactic acid bacteria are those classified as Lactobacillus delbrueckii subspecies bulgaricus.

12. The method according to claim 11, wherein the lactic acid bacteria are Lactobacillus delbrueckii subspecies bulgaricus OLL1073R-1 with accession number FERM BP-10741.


SEQUENCE LISTING (24 sequences)

SEQ ID NO: 1 - DNA, 20 bp, synthetic construct
  misc_feature 1..20: note = Influenza virus M gene region RT-PCR Forward Primer
  source 1..20: mol_type = other DNA
ggcaaatggtacaggcaatg

SEQ ID NO: 2 - DNA, 20 bp, synthetic construct
  misc_feature 1..20: note = Influenza virus M gene region RT-PCR Reverse Primer
  source 1..20: mol_type = other DNA
agcaacgagaggatcacttg

SEQ ID NO: 3 - DNA, 24 bp, synthetic construct
  misc_feature 1..24: note = ZO-1 RT-PCR Forward Primer
  source 1..24: mol_type = other DNA
tggtgtcctacctaattcaactca

SEQ ID NO: 4 - DNA, 24 bp, synthetic construct
  misc_feature 1..24: note = ZO-1 RT-PCR Reverse Primer
  source 1..24: mol_type = other DNA
tgttgatctgaagtgataggtgga

SEQ ID NO: 5 - DNA, 24 bp, synthetic construct
  misc_feature 1..24: note = Occludin RT-PCR Forward Primer
  source 1..24: mol_type = other DNA
acagagcaagatcactatgagaca

SEQ ID NO: 6 - DNA, 23 bp, synthetic construct
  misc_feature 1..23: note = Occludin RT-PCR Reverse Primer
  source 1..23: mol_type = other DNA
cgccagctacaaatattccaaca

SEQ ID NO: 7 - DNA, 22 bp, synthetic construct
  misc_feature 1..22: note = Claudin-1 RT-PCR Forward Primer
  source 1..22: mol_type = other DNA
ccagtcaatgccaggtacgaat

SEQ ID NO: 8 - DNA, 23 bp, synthetic construct
  misc_feature 1..23: note = Claudin-1 RT-PCR Reverse Primer
  source 1..23: mol_type = other DNA
ttggtgttgggtaagaggttgtt

SEQ ID NO: 9 - DNA, 24 bp, synthetic construct
  misc_feature 1..24: note = E-cadherin RT-PCR Forward Primer
  source 1..24: mol_type = other DNA
atagagaacgcattgccacataca

SEQ ID NO: 10 - DNA, 22 bp, synthetic construct
  misc_feature 1..22: note = E-cadherin RT-PCR Reverse Primer
  source 1..22: mol_type = other DNA
ttctgatcggttaccgtgatca

SEQ ID NO: 11 - DNA, 18 bp, synthetic construct
  misc_feature 1..18: note = ICAM-1 RT-PCR Forward Primer
  source 1..18: mol_type = other DNA
gccactgggcaagaacct

SEQ ID NO: 12 - DNA, 18 bp, synthetic construct
  misc_feature 1..18: note = ICAM-1 RT-PCR Reverse Primer
  source 1..18: mol_type = other DNA
tcagtgcggcacgagaaa

SEQ ID NO: 13 - DNA, 22 bp, synthetic construct
  misc_feature 1..22: note = VCAM-1 RT-PCR Forward Primer
  source 1..22: mol_type = other DNA
tgaacccaaacagaggcagagt

SEQ ID NO: 14 - DNA, 23 bp, synthetic construct
  misc_feature 1..23: note = VCAM-1 RT-PCR Reverse Primer
  source 1..23: mol_type = other DNA
ggtatcccatcacttgagccagg

SEQ ID NO: 15 - DNA, 21 bp, synthetic construct
  misc_feature 1..21: note = CEACAM-1 RT-PCR Forward Primer
  source 1..21: mol_type = other DNA
gctggcattgtgattggagta

SEQ ID NO: 16 - DNA, 20 bp, synthetic construct
  misc_feature 1..20: note = CEACAM-1 RT-PCR Reverse Primer
  source 1..20: mol_type = other DNA
ttaggtgggtcattggagtg

SEQ ID NO: 17 - DNA, 20 bp, synthetic construct
  misc_feature 1..20: note = Fibronectin RT-PCR Forward Primer
  source 1..20: mol_type = other DNA
tagccctgtccaggagttca

SEQ ID NO: 18 - DNA, 20 bp, synthetic construct
  misc_feature 1..20: note = Fibronectin RT-PCR Reverse Primer
  source 1..20: mol_type = other DNA
ctgcaagccttcaatagtgt

SEQ ID NO: 19 - DNA, 19 bp, synthetic construct
  misc_feature 1..19: note = PAF-r RT-PCR Forward Primer
  source 1..19: mol_type = other DNA
gcaattgccaggccacaac

SEQ ID NO: 20 - DNA, 21 bp, synthetic construct
  misc_feature 1..21: note = PAF-r RT-PCR Reverse Primer
  source 1..21: mol_type = other DNA
aagggtcacctggtcatggag

SEQ ID NO: 21 - DNA, 21 bp, synthetic construct
  misc_feature 1..21: note = E-selectin RT-PCR Forward Primer
  source 1..21: mol_type = other DNA
agcagagtttcacgttgcagg

SEQ ID NO: 22 - DNA, 19 bp, synthetic construct
  misc_feature 1..19: note = E-selectin RT-PCR Reverse Primer
  source 1..19: mol_type = other DNA
tggcgcagataaggcttca

SEQ ID NO: 23 - DNA, 20 bp, synthetic construct
  misc_feature 1..20: note = beta-actin RT-PCR Forward Primer
  source 1..20: mol_type = other DNA
agaaaatctggcaccacacc

SEQ ID NO: 24 - DNA, 20 bp, synthetic construct
  misc_feature 1..20: note = beta-actin RT-PCR Reverse Primer
  source 1..20: mol_type = other DNA
agaggcgtacagggatagca